(19) United States
(12) Patent Application Publication
(10) Pub. No.: US 2004/0186066 A1
(43) Pub. Date: Sep. 23, 2004

(54) Method of treatment with nelfinavir

(75) Inventors: Carolyn Petersen, San Diego, CA (US); Edward F.C. Pun, San Diego, CA (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, INC.

(21) Appl. No.: 10/776,568

(22) Filed: Feb. 10, 2004

Related U.S. Application Data
(60) Provisional application No. 60/446,444, filed on Feb. 10, 2003.
(60) Provisional application No. 60/524,259, filed on Nov. 21, 2003.

(51) Int. Cl. A61K 31/7056

(52) U.S. Cl. 514/43

(57) ABSTRACT

The invention relates to a method of treating human immunodeficiency virus (HIV) in a mammal comprising administering to a mammal in need thereof a therapeutically effective amount of nelfinavir in a pharmaceutical composition, wherein the nelfinavir is administered with food.

This application claims priority from U.S. Provisional Application Serial No. 60/446,444 filed 10 Feb. 2003, and U.S. Provisional Application Serial No. 60/524,259, filed 21 Nov. 2003, which is hereby incorporated by reference in its entirety.

### FIELD OF THE INVENTION

The invention is directed generally to methods of treating AIDS by administering nelfinavir in combination with food such that the bioavailability of nelfinavir is increased compared to its administration without food.

### BACKGROUND OF THE INVENTION

Human immunodeficiency virus (HIV), the causative agent of AIDS, is a retrovirus that has an integral protease, Type 1 HIV Protease. The HIV protease is important in the maturation of the virus from a noninfectious to an infectious form. Inhibition of the HIV protease prevents post-translational processing such that immature and non-infectious viral particles are released. Several inhibitors of HIV protease are known.

One such inhibitor is [3S-(3R*, 4aR*, 8aR*, 2′S*, 3′S*)]-2-[2′ hydroxy-3′-phenylthiomethyl-4′-aza-5′-oxo-5′-(2″-methyl-3″-hydroxy-phenyl)pentyl]-decahydroiso-quinoline-3-N-t-butylcarboxamide methanesulfonic acid salt; also known as nelfinavir mesylate or nelfinavir and sold by Agouron Pharmaceuticals, Inc. (a Pfizer company) under the trademark Viracept®. Nelfinavir and methods of its manufacture and use are disclosed in the following patents, which are hereby incorporated herein in their entireties by reference: U.S. Pat. Nos. 5,484,926 (issued Jan. 16, 1996), 5,952,343 (issued Sep. 14, 1999), and 6,162,812 (issued Dec. 19, 2000).

Shetty et al (1996) observed that the oral bioavailability of nelfinavir mesylate was 43% in fed rats, dogs and monkeys, but was 29% in animals fasted overnight. Shetty et al, Preclinical pharmacokinetics and distribution to tissue of AG 1343, an inhibitor of human immunodeficiency virus type 1 protease, 40(1) Antimicrob. Agents Chemother. 110, 112 (1996). The fed state consisted of a meal 30 minutes before drug administration. Id. at 111.

Kurowski et al. (2002) reported that nelfinavir administration led to an AUC₀₋₁₂ₕ (plasma concentration integrated over twelve hours) that was 13% less when administered with a light breakfast comprising bread, jam, butter, milk and tea than when administered with a standard breakfast of bread, cheese, butter, milk, cornflakes, yogurt and tea. Kurowski, et al., Limited effect of food consumption on the pharmacokinetics of nelfinavir administered twice daily, 7 Eur. J. Med. Res. 453, 454 (2002). Although the authors consider d this difference statistically significant, they did not consider it clinically relevant. Id. No significant effects of the two different breakfasts were found for the remaining three parameters tested: C_{1-hour post dose}, Cₘₐₓ, and C₁₂₋ₕₒᵤᵣₛ. The light breakfast had 350 kcal including 13 g of fat. The standard breakfast had 800 kcal including 35 g of fat. Id.

Aarnoutse et al. (2003) reported that meal consumption had a significant effect on the AUC_{24h, corr} and Cₘᵢₙ values for nelfinavir and nelfinavir plus M8, the active metabolite of nelfinavir. Aarnoutse, et al., Pharmacokinetics, food intake requirements and tolerability of once-daily combinations of nelfinavir and low-dose ritonavir in healthy volunteers, 55 Br. J. Clin. Pharmacol. 115, 120 (2003). In Aarnoutse et aL. (2003), the full breakfast had 610 kcal, 33% fat (about 22 g), 16% protein (about 24 g), and 51% carbohydrate. Id. at 116. The light breakfast had 271 kcal, 37% fat (about 11 g), 24% protein (about 16 g), and 39% carbohydrates. Id. at 117.

Quart et al. found that single dose administration of nelfinavir under fasting conditions resulted in AUC (area under the plasma concentration-time profile) values that were 27-50% of those observed when the drug was administered with food. Quart et al., Phase I safety, tolerance, pharmacokinetics and food effect studies of AG 1343—a novel protease inhibitor, Natl Conf. Hum. Retroviruses Relat. Infect. (2^{nd}) 167 (1995).

Petersen et al. found that food intake had a marked effect on nelfinavir pharmacokinetics with highest levels achieved after the greatest food intake, that M8 concentrations rose with increasing food intake, but remained at 15-20% of nelfinavir, and that the contribution of different quantities of fat on pharmacokinetics required further study. Petersen et al., Pharmacokinetics of nelfinavir (Viracept® 250 mg tablet): effect of food intake on single-dose PK parameters, 10^{th} Conference on Retroviruses and Opportunistic Infections Abstract 544 (Feb. 10-14, 2003).

The Physician's Desk Reference (PDR) entry for Viracept® nelfinavir (rev. Nov. 2001), recommends that it be administered with food. The PDR reference discloses that the maximum plasma concentrations and AUC were two to three-fold higher under fed conditions compared to fasting. The meals evaluated contained 517 to 759 Kcal, with 153 to 313 Kcal derived from fat. Id. Thus, the advantage of moderate food intake with nelfinavir administration has been shown, but the effect of fat consumption and high caloric intake with nelfinavir has not been well studied.

Some HIV medications have shown strong food effects on bioavailability. Some anti-HIV reverse transcriptase inhibitors are recommended to be administered on an empty stomach, including efavirenz, AZT, ddC, and ddI. Other reverse transcriptase inhibitors can be taken without or with food. Inhibitors of HIV protease vary in their food effects. Indinavir is recommended for administration without food, but with copious amounts of water. In contrast, saquinavir, another HIV protease inhibitor, is recommended for administration with a high fat meal. Amprenavir and lopinavir may be taken without or with food. The differing effect of food on protease inhibitors suggests a lack of a common mechanism underlying the processing and uptake of the protease inhibitors from the gastrointestinal tract.

Optimizing dosaging of protease inhibitors such as nelfinavir is desirable both to minimize side effects and ensure efficacy against HIV. Protease inhibitor therapy is sometimes associated with side effects such as diarrhea, fat redistribution, insulin resistance, diabetes and hyperlipidemia. Lenhard et al., Dietary fat alters HIV protease inhibitor-induced metabolic changes in mice, Am. Soc. Nutr. Sci. 2361 (2000). Yet virological failure of nelfinavir-containing HIV regimens has been related to low plasma levels of nelfinavir. Burger et al., Therapeutic drug monitoring (TDM) of nelfinavir (NELFINAVIR) 1250mg BID in treatment-naive patients improves therapeutic outcome after 1 year: results from ATHENA, 2d Int'l Workshop on Clinical Pharmacology of HIV Therapy, Noordwijk, the Netherlands (2001), Abstract 6.2b. Thus, there is a need for an improved nelfinavir therapy that provides for therapeutic effect while avoiding excessive nelfinavir plasma levels that could lead to undesired side effects.

### SUMMARY OF THE INVENTION

In one aspect, the invention relates a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily the nelfinavir is administered with food and the food comprises more than 800 kcal.

In another aspect, the invention relates a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily the nelfinavir is administered with food and the food comprises more than about 900 kcal or about 1000 kcal.

In a further aspect, the invention relates to the administration of nelfinavir, according to the above-described methods, wherein administration of the nelfinavir occurs between 30 minutes prior to and two hours after consumption of food. The administration of nelfinavir, according to the above-described methods, may also occur between 30 minutes prior to and one hour after consumption of food, or the administration of nelfinavir may occur at about the same time as the consumption of food.

In yet another aspect, the invention relates to a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily the nelfinavir is administered with food and the food comprises more than 800 kcal. In an alternative embodiment, the nelfinavir is administered at least twice daily for at least two weeks and at least twice daily nelfinavir is administered with food and the food comprises more than 800 kcal at each administration.

In still another aspect, the invention relates a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily the nelfinavir is administered with food and the food comprises more than 800 kcal and wherein the food comprises between about 40% fat and about 50% fat by energy content or between about 50% fat and about 60% fat by energy content or between about 60% fat and about 70% fat by energy content or between about 70% fat and about 80% fat by energy content or between about 80% fat and about 90% fat by energy content or between about 90% fat and about 100% fat by energy content. In an alternative embodiment of the above-described method, the food comprises more than 40%, 50%, 60%, 70%, 80% or 90% fat by energy content.

In still another aspect, the invention relates a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily the nelfinavir is administered with food and the food comprises more than 800 kcal and wherein the food comprises from 36 g to 55 g fat or from 40 g to 55 g fat or at least about 55 g fat.

In yet a further aspect, the invention relates a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily the nelfinavir is administered with food and the food comprises more than 800 kcal and wherein the area under the curve from time zero extrapolated to infinite time (AUC(0-∞)) after nelfinavir administration with food is at least about 3-fold greater than the AUC(0-∞) after administration in the fasted state or at least about 5-fold greater than the AUC(0-∞) after administration in the fasted state.

In still a further aspect, the invention relates a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily the nelfinavir is administered with food and the food comprises more than 800 kcal and wherein the mammal is not receiving ritonavir, saquinavir or lopinavir or a stereoisomer, solvate, salt, or prodrug thereof.

In another aspect, the invention relates to a method of treating human immunodeficiency virus (HIV) in a mammal comprising administering orally to a mammal in need thereof a therapeutically effective amount of nelfinavir or pharmaceutically acceptable salt or solvate thereof in a pharmaceutical composition taken with food, wherein the food comprises at least about 500 kcal and at least about 50% fat by energy content.

In a further aspect, the invention relates to a method of treating human immunodeficiency virus (HIV) in a mammal comprising administering orally to a mammal in need thereof a therapeutically effective amount of nelfinavir or pharmaceutically acceptable salt or solvate thereof in a pharmaceutical composition taken with food, wherein the food comprises at least about 500 kcal and at least about 50% fat by energy content and wherein the administration of nelfinavir occurs between 30 minutes prior to and two hours after consumption of food or between 30 minutes prior to and one hour after consumption of food or at about the same time as the consumption of food.

In yet another aspect, the invention relates to a method of treating human immunodeficiency virus (HIV) in a mammal comprising administering orally to a mammal in need thereof a therapeutically effective amount of nelfinavir or pharmaceutically acceptable salt or solvate thereof in a pharmaceutical composition taken with food, wherein the food comprises at least about 500 kcal and at least about 50% fat by energy content and wherein the food comprises between about 50% fat and about 60% fat by energy content or between about 60% fat and about 70% fat by energy content or between about 70% fat and about 80% fat by energy content or between about 80% fat and about 90% fat by energy content or between about 90% fat and about 100% fat by energy content.

In still another aspect, the invention relates to a method of treating human immunodeficiency virus (HIV) in a mammal comprising administering orally to a mammal in need thereof a therapeutically effective amount of nelfinavir or pharmaceutically acceptable salt or solvate thereof in a pharmaceutical composition taken with food, wherein the food comprises at least about 500 kcal and at least about 50% fat by energy content and wherein the food comprises more than about 60%, 70%, 80% or 90% fat by energy content.

In still a further aspect, the invention relates to a method of treating human immunodeficiency virus (HIV) in a mammal comprising administering orally to a mammal in need thereof a therapeutically effective amount of nelfinavir or pharmaceutically acceptable salt or solvate thereof in a pharmaceutical composition taken with food, wherein the food comprises at least about 500 kcal and at least about 50% fat by energy content and wherein the food comprises from 36 g to 55 g fat or from 40 g to 55 g fat or at least about 55 g fat.

In still another aspect, the invention relates to a method of treating human immunodeficiency virus (HIV) in a mammal comprising administering orally to a mammal in need thereof a therapeutically effective amount of nelfinavir or pharmaceutically acceptable salt or solvate thereof in a pharmaceutical composition taken with food, wherein the food comprises at least about 500 kcal and at least about 50% fat by energy content and wherein the food comprises at least about 600 kcal or at least about 700 kcal or at least about 800 kcal or at least about 900 kcal or at least about 1000 kcal.

In yet a further aspect, the invention relates to a method of treating human immunodeficiency virus (HIV) in a mammal comprising administering orally to a mammal in need thereof a therapeutically effective amount of nelfinavir or pharmaceutically acceptable salt or solvate thereof in a pharmaceutical composition taken with food, wherein the food comprises at least about 500 kcal and at least about 50% fat by energy content and wherein the area under the curve from time zero extrapolated to infinite time (AUC(0-∞)) after nelfinavir administration with food is at least about 3-fold greater than the AUC(0-∞) after administration in the fasted state.

In yet another aspect, the invention relates to a method of treating human immunodeficiency virus (HIV) in a mammal comprising administering orally to a mammal in need thereof a therapeutically effective amount of nelfinavir or pharmaceutically acceptable salt or solvate thereof in a pharmaceutical composition taken with food, wherein the food comprises at least about 500 kcal and at least about 50% fat by energy content and wherein the area under the curve from time zero extrapolated to infinite time (AUC(0-∞)) after nelfinavir administration with food is at least about 5-fold greater than the AUC(0-∞) after administration in the fasted state.

In a further aspect, the invention relates to a method of treating human immunodeficiency virus (HIV) in a mammal comprising administering orally to a mammal in need thereof a therapeutically effective amount of nelfinavir or pharmaceutically acceptable salt or solvate thereof in a pharmaceutical composition taken with food, wherein the food comprises at least about 500 kcal and at least about 50% fat by energy content and wherein the mammal is not receiving ritonavir, saquinavir or lopinavir or a stereoisomer, solvate, salt, or prodrug thereof.

In yet another aspect, the invention relates to a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily nelfinavir is taken with food and the food comprises more than about 500 kcal and more than about 50% fat by energy content.

In a further aspect, the invention relates to a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily nelfinavir is taken with food and the food comprises more than about 500 kcal and more than about 50% fat by energy content and wherein the administration of nelfinavir occurs between 30 minutes prior to and two hours after consumption of food or between 30 minutes prior to and one hour after consumption of food or the administration of nelfinavir occurs at about the same time as the consumption of food.

In a further aspect, the invention relates to a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily nelfinavir is taken with food and the food comprises more than about 500 kcal and more than about 50% fat by energy content and wherein nelfinavir is administered at least twice daily for at least two weeks and at least twice daily nelfinavir is administered with food and the food comprises more than 500 kcal, 600 kcal, 700 or 900 kcal (and more than about 50% fat by energy content at each administration).

In a further aspect, the invention relates to a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily nelfinavir is taken with food and the food comprises more than about 500 kcal and more than about 50% fat by energy content and wherein the food comprises between about 50% fat and about 60% fat by energy content, or between about 60% fat and about 70% fat by energy content, or between 70% fat and about 80% fat by energy content, or between about 80% fat and about 90% fat by energy content, or between about 90% fat and about 100% fat by energy content.

In still a further aspect, the invention relates to a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily nelfinavir is taken with food and the food comprises more than about 500 kcal and more than about 50% fat by energy content and wherein the food comprises more than about 60%, 70%, 80% or 90% fat by energy content.

In a further aspect, the invention relates to a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily nelfinavir is taken with food and the food comprises more than about 500 kcal and more than about 50% fat by energy content and wherein the food comprises from 36 g to 55 g fat or from 40 g to 55 g fat or the food comprises at least about 55 g fat.

In a further aspect, the invention relates to a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily nelfinavir is taken with food and the food comprises more than about 500 kcal and more than about 50% fat by energy content and wherein the mammal is not receiving ritonavir, saquinavir or lopinavir or a stereoisomer, solvate, salt or prodrug thereof.

In yet another aspect, the invention relates to a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily nelfinavir is taken with food and the food comprises more than about 500 kcal and more than about 50% fat by energy content and wherein the area under the curve from time zero extrapolated to infinite time (AUC(0-∞)) after nelfinavir administration with food is at least about 3-fold greater than the AUC(0-∞) after administration in the fasted state.

In a further aspect, the invention relates to a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily nelfinavir is taken with food and the food comprises more than about 500 kcal and more than about 50% fat by energy content and wherein the area under the curve from time zero extrapolated to infinite time (AUC(0-∞)) after nelfinavir administration with food is at least about 5-fold greater than the AUC(0-∞) after administration in the fasted state.

In a further aspect, the invention relates to a method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily nelfinavir is taken with food and the food comprises more than about 500 kcal and more than about 50% fat by energy content and wherein the mammal is not receiving ritonavir, saquinavir or lopinavir or a stereoisomer, solvate, salt, or prodrug thereof.

In another aspect, the invention relates to a kit comprising a therapeutically effective oral dose of nelfinavir and a printed material comprising instructions for administering the dose with food comprising at least 800 kcal in a high-fat meal.

In a further aspect, the invention relates to a kit comprising a therapeutically effective oral dose of nelfinavir and a printed material comprising instructions for administering the dose with food comprising at least 800 kcal in a high-fat meal and wherein the label further comprises instructions for administering the dose with food comprising at least 50% fat by energy content

In yet another aspect, the invention relates to a kit comprising a therapeutically effective oral dose of nelfinavir and a printed material comprising instructions for administering the dose with food comprising at least 800 kcal in a high-fat meal and wherein the high-fat meal is recited to comprise more than about 36 g of fat.

In another aspect, the invention relates to a therapeutic composition for the treatment of human immunodeficiency virus (HIV) in a mammal comprising fat and a therapeutically effective amount of nelfinavir in a weight ratio of at least about 25 fat:1 nelfinavir.

In a further aspect, the invention relates to a therapeutic composition for the treatment of human immunodeficiency virus (HIV) in a mammal comprising fat and a therapeutically effective amount of nelfinavir in a weight ratio of at least about 25 fat:1 nelfinavir and wherein the weight ratio is greater than about 30 fat:1 nelfinavir

In yet another aspect, the invention relates to a therapeutic composition for the treatment of human immunodeficiency virus (HIV) in a mammal comprising fat and a therapeutically effective amount of nelfinavir in a weight ratio of at least about 25 fat:1 nelfinavir and wherein the amount of nelfinavir is between about 100 mg and about 1500 mg.

### BRIEF DESCRIPTION OF THE FIGURES

FIG. 1. Mean nelfinavir plasma concentration-time profiles following administration of 1250-mg oral doses to fasting subjects (closed circles), with a low calorie/low fat meal (open circles), with a moderate calorie/low fat meal (closed squares), and with a high calorie/high fat meal (open squares). Upper panel and lower panel use linear and semi-logarithmic plots, respectively.

FIG. 2. Individual nelfinavir Cm, (upper panel) and AUC(0-∞) (lower panel) values following administration of 1250-mg nelfinavir oral doses to fasting subjects (0 Kcal), with a low calorie/low fat meal (125 Kcal), with a moderate calorie/low fat meal (500 Kcal), and with a high calorie/high fat meal (1000 Kcal). Individual subject and mean values are illustrated by numbers and triangles, respectively.

FIG. 3. Mean nelfinavir plasma concentration as a function of meal caloric content. The AUC(0-∞) (lozenge, solid line) values are in units of μg·hr/mL and have a correlation of r²>0.97. The Cₘₐₓ (squares, dashed line) values are in units of μg/mL and have a correlation of r²>0.89. Measurement followed administration of a 1250 mg oral dose of nelfinavir.

FIG. 4. Mean nelfinavir plasma concentrations as a function of meal protein content. The AUC(0-∞) (lozenge, solid line) values are in units of μg·hr/mL and have a correlation of r²>0.99. The Cₘₐₓ (squares, dashed line) values are in units of μg/mL and have a correlation of r²>0.96. Measurement followed administration of a 1250 mg oral dose of nelfinavir.

FIG. 5. Mean simulated nelfinavir steady-state plasma concentration-time profiles following BID administration of 1250-mg oral doses to fasting subjects (closed circles), with a low calorie/low fat meal (open circles), with a moderate calorie/low fat meal (closed squares), and with a high calorie/high fat meal (open squares). The bars represent standard errors.

FIG. 6. Mean M8 plasma concentration-time profiles following administration of 1250-mg nelfinavir oral doses to fasting subjects (closed circles), with a low calorie/low fat meal (open circles), with a moderate calorie/low fat meal (closed squares), and with a high calorie/high fat meal (open squares). Upper and lower panels represent linear and semi-logarithmic plots, respectively.

FIG. 7. Individual M8 Cₘₐₓ (upper panel) and AUC(0-∞) (lower panel) values following administration of 1250-mg nelfinavir oral doses to fasting subjects (0 Kcal), with a low calorie/low fat meal (125 Kcal), with a moderate calorie/low fat meal (500 Kcal), and with a high calorie/high fat meal (1000 Kcal). Individual subject and mean values are illustrated by numbers and triangles, respectively.

FIG. 8. Mean nelfinavir plasma concentration-time profiles following administration of 5×250-mg nelfinavir tablets to fasting subjects (filled circles), during a moderate calorie/low fat meal (open circles), and during a moderate calorie/high fat meal (filled squares), according to Example 2. Upper and lower panels are linear and semi-logarithmic plots, respectively.

FIG. 9. Individual nelfinavir Cₘₐₓ (upper panel) and AUC(0-∞) values (lower panel) following administration of 5×250-mg nelfinavir tablets to fasting subjects, during a moderate calorie/low fat meal, and during a moderate calorie/high fat meal, according to Example 2. Individual and mean values are represented by open circles and triangles, respectively.

FIG. 10. Mean M8 plasma concentration-time profiles following administration of 5×250-mg nelfinavir tablets to fasting subjects (filled circles), during a moderate calorie/low fat meal (open circles), and during a moderate calorie/high fat meal (filled squares) according to Example 2. Upper and lower panels are linear and semi-logarithmic plots, respectively.

FIG. 11. Individual M8 Cₘₐₓ (upper panel) and AUC(0-∞) values (lower panel) following administration of 5×250-mg nelfinavir tablets to fasting subjects, during a moderate calorie/low fat meal, and during a moderate calorie/high fat meal according to Example 2. Individual and mean values are represented by open circles and triangles, respectively.

### DETAILED DESCRIPTION OF THE INVENTION

In one aspect the invention relates to a method of treating HIV in a mammal comprising administering to a mammal in need thereof a therapeutically effective amount of nelfinavir in a pharmaceutical composition with food and the food comprises more than 800 kcal. Alternatively, the food may comprise more than about 900 kcal or more than about 1000 kcal. The mammal preferably is a human.

Preferably, under the methods of the invention, HIV is treated by administering nelfinavir at least once daily for at least two weeks. More preferably, nelfinavir is administered at least twice daily. Other preferable conditions of treatment include nelfinavir administration three times daily. Preferably, nelfinavir therapy continues for at least two weeks. More preferably, nelfinavir is administered for at least four weeks. Other durations of treatment that are preferred are at least three months, at least six months, and at least one year.

Administration of nelfinavir should be with food. Preferably, nelfinavir is administered between 30 minutes prior to and two hours after consumption of food. More preferably, nelfinavir is administered between 30 minutes prior to and one hour after consumption of food. Still more preferably, the administration of nelfinavir occurs at about the same time as the consumption of food. Preferably, nelfinavir is administered at least once a day with one of the meals described herein. More preferably, nelfinavir is administered at least twice a day with each administration of nelfinavir being with one of the meals described herein. Also preferable is administration of nelfinavir three times a day with each administration of nelfinavir being with one of the meals described herein.

One of the preferred meals of the invention is at least 800 kcal. More preferably, nelfinavir is administered with food of at least 800 kcal and one of the following ranges of fat content as measured by percentage of energy content: between about 40% fat and about 50% fat, between about 50% fat and about 60% fat, between about 60% fat and about 70% fat, between about 70% fat and about 80% fat, between about 80% fat and about 90% fat and between about 90% fat and about 100% fat. Also preferable is administration of nelfinavir with a meal of at least 800 kcal and at least one of the following levels of fat content as measured by percentage of energy content: more than 40% fat, more than 50% fat, more than 60% fat, more than 70% fat, more than 80% fat and more than about 90% fat. Also, nelfinavir may be administered with food comprising at least 800 kcal and an amount of fat from the following list: from 36 g to 55 g fat, from 40 g to 55 g fat and at least about 55 g fat.

Another method of the invention is treating human immunodeficiency virus (HIV) in a mammal comprising administering orally to a mammal in need thereof a therapeutically effective amount of nelfinavir in a pharmaceutical composition taken with food, wherein the food comprises at least about 500 kcal and at least about 50% fat by energy content. Preferably, the food comprises at least about 500 kcal and has a fat content as measured by percentage of energy content from one of the following ranges: between about 50% fat and about 60% fat, between about 60% fat and about 70% fat, between about 70% fat and about 80% fat, between about 80% fat and about 90% fat, and between about 90% fat and about 100% fat. Also preferable is administration of nelfinavir with a meal of at least about 500 kcal and at least one of the following levels of fat content as measured by percentage of energy content: more than about 60% fat, more than about 70% fat, more than about 80% fat and more than about 90% fat. Also, nelfinavir may be administered with food comprising at least about 500 kcal and an amount of fat from the following list: from 36 g to 55 g fat, from 40 g to 55 g fat and at least about 55 g fat. Also preferable is administration of nelfinavir with food wherein the food comprises at least about 50% fat by energy content and at least 600 kcal, at least 700 kcal, at least 800 kcal, at least 900 kcal, or at least 1000 kcal.

In the methods of the invention, administration of a pharmaceutical composition of nelfinavir with food results in an increase in plasma concentration of nelfinavir. The plasma concentration can be measured as AUC. Preferably, the inventive methods result in an increase in the area under the curve from time zero extrapolated to infinite time (AUC(0-∞)) after nelfinavir administration with food that is at least about 3-fold greater than the AUC(0-∞) after administration in the fasted state and, more preferably, is at least about 5-fold greater than the AUC(0-∞) after administration in the fasted state.

In one aspect, the plasma concentration can be measured as Cₘₐₓ. The method can further comprise increasing Cₘₐₓ values at least about 3-fold compared to a fasted subject.

In still yet another aspect of the method of the invention, administration of the nelfinavir composition with food as described herein increases plasma concentration of a metabolite of nelfinavir, hydroxyl-t-butylamide, also called M8. The method can further comprise increasing AUC values of M8 at least about 3-fold compared to a fasted subject and more preferably at least 5-fold.

The amount of the nelfinavir administered can be any therapeutically effective amount. For example, for an adult a dose of 1250 mg twice daily or 750 mg three times daily is recommended. In pediatric patients, an effective dose is 20-30 mg/kg three times daily. Nelfinavir can be administered in any pharmaceutically acceptable form, such as a salt, stereoisomer, solvate or prodrug of nelfinavir.

In another aspect of the invention, a composition comprising nelfinavir is administered to a subject to whom no other HIV medications are administered. In a particular aspect, a composition comprising nelfinavir is administered to a subject who is not receiving ritonavir, saquinavir or lopinavir or a stereoisomer, solvate, salt, or prodrug thereof.

In yet another aspect, nelfinavir is administered to a subject suffering from an HIV infection who is receiving at least one other HIV medication including, but not limited to a protease inhibitor, a nucleoside analogue reverse transcriptase inhibitor, a non-nucleoside reverse transcriptase inhibitor, a nucleotide analogue reverse transcriptase inhibitor, or a viral fusion inhibitor. The additional HIV medication can be, but is not limited to, one or more of the following drugs: Retrovir® (3′-azido-2′,3′-dideoxythymidine or AZT), Epivir® (2′,3′-dideoxy 3′-thiacytidine or 3TC), Combivir® (AZT in combination with 3TC), Videx® (2′,3′-dideoxyinosine or didanosine or ddI), Hivid® (2′,3′-dideoxycytidine or ddC), Zerit® (stavudine or 2′,3′-didehydro-3′-deoxythymidine or 3′-deoxythymidin-2′-ene or d4T), Ziagen® (abacavir), Viramun® (nevirapine), Rescriptor® (delavirdine), Sustiva® (efavirenz), Preveon® (adefovir dipovoxil), Crixivan® (indinavir), Angenerase® (amprenavir) and Hydrea® (hydroxy urea).

The invention also relates to a kit comprising a therapeutically effective oral dose of nelfinavir and printed material comprising instructions for administering the dose with food according to one of the methods of the invention. For example, the printed material may comprise instructions that nelfinavir be administered with food comprising at least 800 kcal in a high-fat meal. The high-fat meal preferably is instructed to comprise at least 40% fat by energy content. Alternatively, the printed material may instruct that nelfinavir be administered with food comprising at least 500 kcal and 50% fat by energy content. In another embodiment, the printed material may instruct that the food comprise more than about 36 g of fat.

In still yet another aspect, the invention relates to a therapeutic composition for the treatment of HIV comprising fat and a therapeutically effective amount of nelfinavir in a weight ratio of at least about 25 fat: 1 nelfinavir. Also preferred is a composition in which the weight ratio is greater than about 30 fat: 1 nelfinavir. Preferably, the amount of nelfinavir is between about 100 mg and about 1500 mg, more preferably between 250 mg to 625 mg inclusive.

### EXAMPLE 1

### Evaluation of Total Kilocalories and Fat on Nelfinavir Bioavailability

A phase I, randomized, open-label crossover study to evaluate the impact of total kilocalories and fat content on single-dose pharmacokinetic parameters of the nelfinavir 250 mg tablet formulation in normal healthy volunteers was performed.

Methods

Healthy volunteers entered the study and received the following treatment in random order at least 1 week apart:

1) 5×250 mg nelfinavir tablets, fasted

2) 5×250 mg nelfinavir tablets (breakfast meal 1, 125 Kcal/20% fat=low cal/low fat)

3) 5×250 mg nelfinavir tablets (breakfast meal 2, 500 Kcal/20% fat=medium cal/low fat)

4) 5×250 mg nelfinavir tablets (breakfast meal 3, 1000 Kcal/50% fat=high cal/high fat).

Plasma concentrations of nelfinavir and its active hydroxy-t-butylamide metabolite (M8) were measured by validated high performance liquid chromatography (HPLC) methods. Pharmacokinetic parameters were determined from plasma concentration-time data using standard methods.

The following statistical methods were used:

1) Log-transformed nelfinavir area under the concentration-time profile (AUC) was the primary parameter analyzed to determine the effect of caloric and fat content of meals on nelfinavir pharmacokinetics.

2) Secondary parameters included were nelfinavir T_{1/2}, time to maximum observed plasma concentration (Tₘₐₓ), and log transformed Cₘₐₓ, as well as M8 pharmacokinetic parameters.

3) Parameter values were evaluated by Analysis of Variance (ANOVA) using a model incorporating sequence, subject within sequence, period and treatment effects. Statistical tests were performed using the Type III sum of squares derived using WinNONlin Pro Version 2.1. Least squares treatment mean values were determined for each parameter.

4) Results from ANOVA were used to calculate 90% confidence intervals for the ratio (test/reference) least-square treatment mean values, where administration of single nelfinavir doses in the fasting state was the reference treatment.

Menus for the standardized breakfast meals were as follows. Nutrient composition data is from the USDA Nutrient Database for Standard Reference, Release 14 and select manufacturer's data for specific brands.

| | | | | | |
|---|---|---|---|---|---|
| | | Energy, | Fat, | Protein, | Carbo., |
| Food | Amount | Kcal | gm | gm | gm |

Breakfast M al 1: 125 Kcal, 10 gm proteins, 3 gm fat (20%)

| | | | | | |
|---|---|---|---|---|---|
| Milk, 1% | 10 fluid | 125 | 3.1 | 10.0 | 14.6 |
| | ounces | | | | |

Breakfast Meal 2: 500 Kcal, 20 gm protein, 11 gm fat (20%)

| | | | | | |
|---|---|---|---|---|---|
| Orange Juice | 8 fluid | 110 | 0.1 | 1.7 | 26.8 |
| | ounces | | | | |
| Yogurt, Dannon | 4 ounces | 60 | 0 | 5.0 | 26 |
| Light and Fit | | | | | |
| Cereal, Cherrios | 1 cup | 110 | 1.8 | 3.1 | 22.9 |
| Milk, 2% | 8 fluid | 122 | 4.6 | 8.0 | 11.7 |
| | ounces | | | | |
| Toast, wheat | 1 slice | 65 | 1 | 2.7 | 11.8 |
| Butter | 1 teaspoon | 34 | 3.8 | 0 | 0 |
| Total | | 501 | 11.3 | 20.5 | 99.2 |

Breakfast Meal 3: 1000 Kcal, 36 gm protein, 56 gm fat (50%)

| | | | | | |
|---|---|---|---|---|---|
| Eggs fried in | 2 extra large | 172 | 11.6 | 14.5 | 1.4 |
| Butter | 2 teaspoons | 68 | 7.6 | 0 | 0 |
| Bacon | 3 strips | 108 | 9.3 | 5.9 | 0 |
| Toast, wheat | 2 slices | 130 | 2 | 5.4 | 23.6 |
| Butter | 2.5 | 85 | 9.5 | 0 | 0 |
| | teaspoons | | | | |
| Hash Brown | 4 ounces | 105 | 0 | 2 | 23 |
| Potatoes, | 1½ cups | 68 | 7.6 | 0 | 0 |
| ORE IDA | 2 teaspoons | | | | |
| frozen, | | | | | |
| southern | | | | | |
| style OR | | | | | |
| shredded | | | | | |
| Cooked in | | | | | |
| Butter | | | | | |
| Milk, whole | 8 fluid ounces | 149 | 8.1 | 8.0 | 11.4 |
| 3.25% | | | | | |
| Orange juice | 8 fluid ounces | 112 | 0.1 | 1.7 | 26.8 |
| Total | | 997 | 55.8 | 37.5 | 86.2 |

All subjects who were included in the study were willing to adhere to the specified restrictions, were between 18 and 60 years of age (inclusive), had a Body Mass Index (BMI) between 18 and 31 kg/M² (inclusive), and were HIV-1 and HIV-2 seronegative. All females were not pregnant, as determined by a serum pregnancy test prior to Day 1 and a urine pregnancy test prior to each dose.

All subjects received oral doses of nelfinavir according to the schedule in Table 1.

TABLE 1
Dosing Schedule for Nelfinavir

| | | | Duration of | Route of |
|---|---|---|---|---|
| Treatment | Dose | Dosing Regimen | Treatment | Administration |
| Nelfinavir tablet | 1250-mg | Single dose | Days 1, 8, 15, | Oral |
| | (5 × 250-mg) | | and 22 | |

Nelfinavir was administered as five 250-mg tablets with 240 mL of water. All subjects received a standardized snack the evening they were admitted to the in-patient facility. For the fasting evaluation, subjects were required to complete an overnight fast of at least 10 hours prior to dosing in the morning. For the fed pharmacokinetic evaluations, subjects were required to complete an overnight fast of at least 10 hours, prior to receiving the protocol-specific standardized breakfast meal (that is, Meal 1, Meal 2, or Meal 3). Subjects were given 30 minutes to complete their standardized breakfast meal. Dosing was performed in the morning, immediately following the subject's completion of the standardized breakfast meal and after the subject's pre-dose pharmacokinetic specimen had been collected. Subjects could not ingest water 1 hour prior to or 1 hour after dosing. A standardized lunch was given at least 4 hours after the morning dose and a standardized dinner was given at least 10 hours after the morning dose.

Subjects received nelfinavir according to a randomized schedule. Randomization codes, subject identifiers, and assigned treatments were provided to each investigator.

Subjects were to refrain from strenuous exercise within 48 hours prior to any clinical laboratory or pharmacokinetic evaluations.

In addition, subjects were to refrain from consuming alcohol, starting 48 hours before each dose and continuing 12 hours following each dose and abstain from grapefruit and products containing grapefruit for 7 days prior to study entry (Day 1) and continuing through study completion.

Twenty-four subjects entered the study and twenty subjects completed the study.

Pharmacokinetics

For pharmacokinetic sampling, blood samples, 5 mL each, were collected into heparinized vacuum tubes (green top tubes) via an indwelling catheter or direct venipuncture. The actual time of each collection was recorded on the source document. The timing of each sample collection was as follows: predose and at 0.5, 1, 2, 3, 4, 6, 8, and 12 hours postdose on Days 1, 8,15, and 22.

All blood samples were kept at 4° C. (using either ice or cryoblock) until centrifugation. Blood samples were centrifuged within 1 hour of collection, at approximately 1000×g for 15 minutes, to separate the plasma. The plasma samples were split evenly into 2 aliquots and stored in appropriately labeled polypropylene transport tubes. Plasma was stored frozen at −20° C. or lower until analysis.

Sample analysis conditions for nelfinavir and M8 in plasma are summarized in Table 2.

TABLE 2
Summary of Sample Analysis for Nelfinavir and M8 in Human Plasma
(Example 1)
Method Description

| | |
|---|---|
| Matrix | Plasma (Sodium Heparin) |
| Type of Method | HPLC |
| Deviations From Validated Method | None |
| Sample Volume | 250 μL |
| Internal Standard | ALD-126462 |

Study Assay Performance

| | Analytical Range | | Quality Control Samples | |
|---|---|---|---|---|
| | Lower Limit | Upper Limit | Precision | Accuracy |
| Analyte | (LLOQ) | (ULOQ) | (% CV) | (% RE) |
| Nelfinavir | 0.0500 μg/mL | 10.0 μg/mL | ≦7.31% | 2.94 to +3.91% |
| M8 | 0.0500 μg/mL | 10.0 μg/mL | ≦4.23% | 4.07 to 5.55% |

Sample Handling

| | |
|---|---|
| Storage Conditions | −20° C. |
| Stability Under Storage Conditions | 656 days |
| Stability ≧ Longest Time From Collection | Yes |
| to Analysis | |

Pharmacokinetic parameter values were calculated using WinNonlin Pro Version 2.1. Pharmacokinetic parameters determined in this study are given in Table 3.

TABLE 3
Pharmacokinetic Parameters

| Parameter | Definition | Method of Determination |
|---|---|---|
| Cmax | Maximum plasma concentration | Observed |
| Tmax | Time for Cmax | Observed |
| AUC(0-tlqc) | Area under plasma concentration-time | Linear trapezoidal method |
| | profile from time zero to time for the last | |
| | quantifiable concentration (lqc) | |
| C₁₂ | Concentration at 12 hours postdose | Observed |
| λz | Terminal rate constant | Absolute value of slope of linear |
| | | regression of natural logarithm |
| | | (ln) of concentration on time |
| | | during the terminal phase of |
| | | concentration-time profile |
| t1/2 | Terminal half-life | ln(2)/λz |
| AUC(0-∞) | Area under plasma concentration-time | AUC(0-tlqc) + lqc/λz |
| | profile from time zero extrapolated to | |
| | infinite time | |
| AUCextrap | Percent AUC(0-∞) due to extrapolation | 100% · lqc/[λz · AUC(0-∞)] |

Descriptive statistics of nelfinavir Cmax and AUC, as well as C₁₂ values were examined to determine the effect of meals of various Kcal content on the variability of these parameter values.

Individual nelfinavir plasma concentrations were used to predict steady-state plasma concentrations during BID administration with meals of various Kcal content. WinNonlin Pro Version 3.2 noncompartmental superposition was used for this simulation. Predicted steady-state Cmax and Cmin values were compared.

The food effect on nelfinavir was estimated by the log-difference between the AUC and Cmax observed at different Kcal levels and the AUC and Cmax observed under the fasted condition. The primary endpoint of this study is the log-difference in AUC between each of the caloric intake groups as compared to the fasted condition.

Based on the results of a 2×2 crossover study for 625 mg bioequivalence under the fasting condition, the Root Mean Square Error for log-AUC is 0.4 from the crossover ANOVA model. A 35% difference in mean AUC between two Kcal levels was equivalent to a 0.3 mean difference in log-AUC. With 5% Type I error and a two side test, 21 subjects in the study should have 90% power in detecting a 35% difference in mean AUC between any of the two caloric levels.

Pharmacokinetic Results

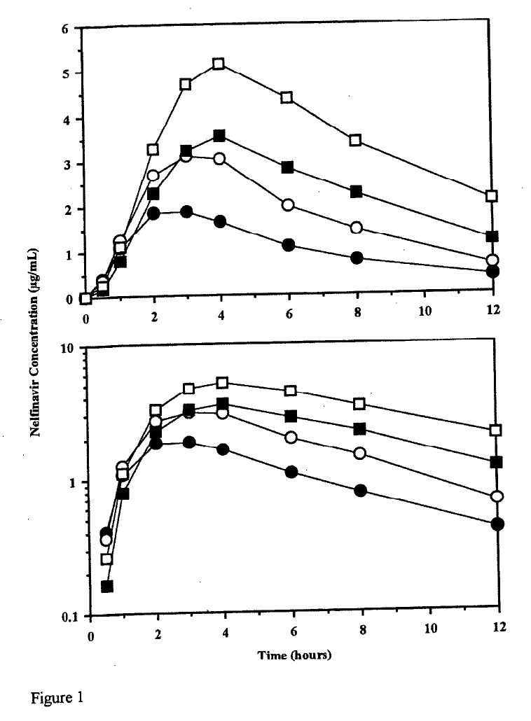

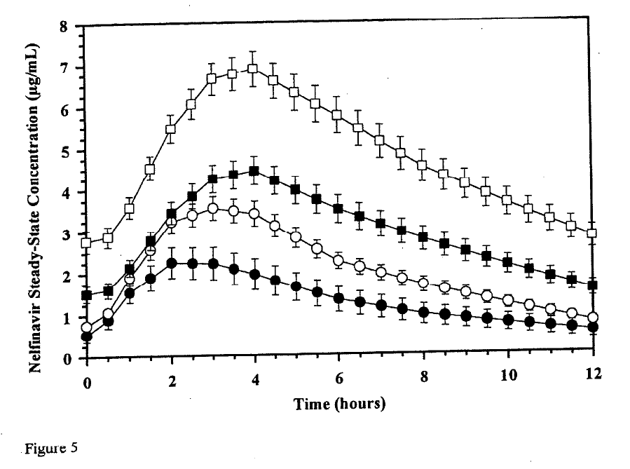

Pharmacokinetic parameter values in the comparison of nelfinavir administration with test meals relative to those to fasting subjects are summarized in Table 4 and reported in FIGS. 1-7.

TABLE 4
Summary of Nelfinavir Pharmacokinetic Parameter Values Following
Administration of 1250 mg Nelfinavir Oral Doses to Fasting Subjects
(Reference) or to Subjects taking Nelfinavir with a Meal.

| | Least-Squares | | | |
|---|---|---|---|---|
| | Mean Values | | | |
| | | with | | 90% |
| | Fasting | Meal | | Confidence |
| Parameter | (Reference) | (Test) | Ratio | Interval |

Low Calorie/Low Fat Meal

| | | | | |
|---|---|---|---|---|
| N | 22 | 21 | | |
| Cmax, μg/mL | 1.57 | 3.16 | 201 | 163 to 249 |
| tmax, hr | 2.18 | 3.02 | 139 | Not Applicable |
| AUC(0-tlqc), μg · hr/mL | 9.04 | 20.0 | 221 | 173 to 283 |
| AUC(0-∞), μg · hr/mL | 10.6 | 23.1 | 218 | 166 to 285 |
| t1/2, hr | 4.10 | 3.59 | 87.5 | 69.4 to 106 |

Moderate Calorie/Low Fat Meal

| | | | | |
|---|---|---|---|---|
| N | 22 | 22 | | |
| Cmax, μg/mL | 1.57 | 3.67 | 234 | 190 to 288 |
| tmax, hr | 2.18 | 3.87 | 178 | Not Applicable |
| AUC(0-tlqc), μg · hr/mL | 9.04 | 25.5 | 282 | 222 to 358 |
| AUC(0-∞), μg · hr/mL | 10.6 | 33.4 | 314 | 241 to 409 |
| t1/2, hr | 4.10 | 4.77 | 116 | 98.7 to 134 |

High Calorie/High Fat Meal

| | | | | |
|---|---|---|---|---|
| N | 22 | 23 | | |
| Cmax, μg/mL | 1.57 | 5.20 | 331 | 270 to 406 |
| tmax, hr | 2.18 | 3.98 | 183 | Not Applicable |
| AUC(0-tlqc), μg · hr/mL | 9.04 | 38.9 | 430 | 340 to 543 |
| AUC(0-∞), μg · hr/mL | 10.6 | 55.3 | 520 | 402 to 674 |
| t1/2, hr | 4.10 | 5.63 | 139 | 121 to 156 |

Ratio = Ratio of treatment mean values, expressed as a percentage (100% × test/reference).
90% Confidence Interval = 90% confidence interval estimate for the ratio (test/reference) of treatment mean values, expressed as a percentage of the reference mean.

Thus, based on area under the plasma concentration-time profile from time zero extrapolated to infinite time (AUC(0-∞)) values, the bioavailability of nelfinavir was 2.2-, 3.1-, and 5.2-fold higher following administration with meals 1, 2, and 3 respectively, relative to that in fasting subjects.

Administration of nelfinavir with meals of increasing caloric content resulted in longer nelfinavir time to maximum observed plasma concentration (tmax) values and higher Cmax values. Mean tmax values were approximately 1, 1.5, and nearly 2 hours longer when administered with a low, moderate, and high calorie meal, respectively, relative to that in fasting subjects; mean Cmax values were 2-, 2.3-, and 3.3-fold higher, respectively. In general, caloric content did not have a profound effect on nelfinavir half-life (t½) values with mean values ranging from 3.6 to 5.6 hours.

As shown in FIG. 1, nelfinavir C₁₂ values in subjects receiving the dose with the high calorie/high fat meal were higher than Cmax values in fasting subjects. Additionally, administration with meals decreases variability in nelfinavir plasma concentrations. The mean and coefficient of variation (% CV) nelfinavir C₁₂ values were as follows:

| | |
|---|---|
| Fasting | 0.41 μg/mL (121%) |
| 125 Kcal/20% fat | 0.65 μg/mL (55%) |
| 500 Kcal/20% fat | 1.19 μg/mL (51%) |
| 1000 Kcal/50% fat | 2.07 μg/mL (42%). |

TABLE 5
Summary of Pharmacokenetic Results

| | Food Intake (kcal/fat) | | | |
|---|---|---|---|---|
| PK Parameter | Fasting | 125/20% | 500/20% | 1000/50% |
| AUC₁₂, μg · hr/mL | 9.04 | 20.0 (2.2X) | 25.5 (2.8X) | 38.9 (4.3X) |
| (×fasting) | | | | |
| 90% CL, ×fasting | | 1.73-2.83X | 2.22-3.58X | 3.40-5.43X |
| AUCinf, μg · hr/mL | 10.6 | 23.1 (2.2X) | 33.4 (3.1X) | 55.3 (5.2X) |
| (×fasting) | | | | |
| 90% CL, ×fasting | | 1.66-2.85X | 2.14-4.09X | 4.02-6.74X |
| Cmax, μg · hr/mL | 1.57 | 3.16 (2.0X) | 3.67 (2.3X) | 5.20 (3.3X) |
| (×fasting) | | | | |
| 90% CL, ×fasting | | 1.63-2.49X | 1.90-2.88X | 2.70-4.06X |
| M8 AUCinf/ | 26.5 | 15.8 | 15.3 | 20.8 |
| NFV AUCinf (%) | | | | |

Mean plasma nelfinavir concentration-time profiles for each treatment are depicted in FIG. 1. Mean nelfinavir pharmacokinetic parameter values in the comparison of nelfinavir administration with test meals relative to those to fasting subjects are presented in the following tables:

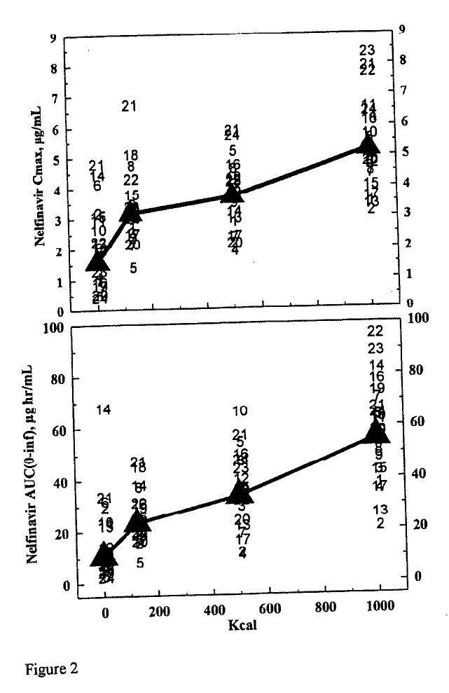

FIG. 2 depicts individual Cmax and AUC(0-∞) values following administration of an 1250 mg oral dose of nelfinavir, as a function of the energy content of the accompanying meal, if any.

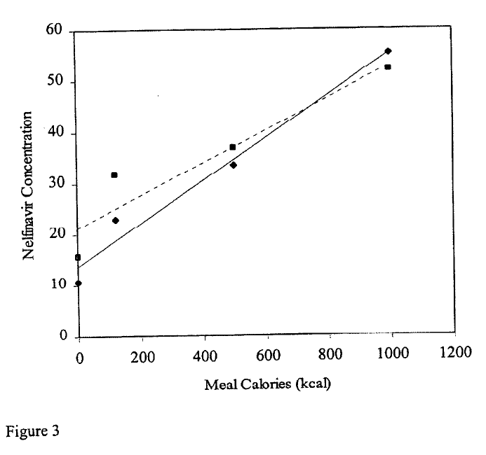

The average nelfinavir plasma concentration is depicted in FIG. 3 as a function of the caloric value of the accompanying meal. The solid line represents AUC(0-∞)(r²>0.97) and the dashed line represents Cmax (r²<0.95).

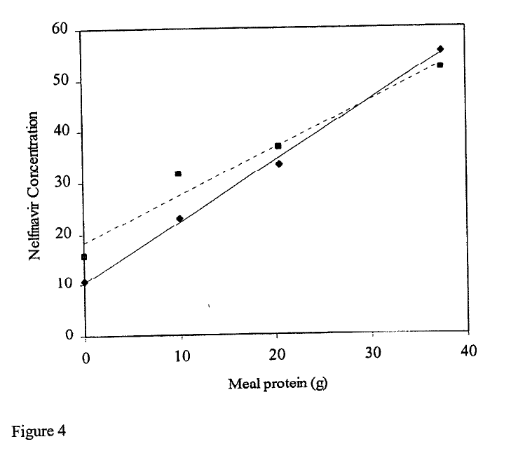

The average nelfinavir plasma concentration is depicted in FIG. 4 as a function of the protein content of the accompanying meal, if any. The solid line represents AUC(0-∞)(r²>0.99) and the dashed line represents Cmax (r²<0.95).

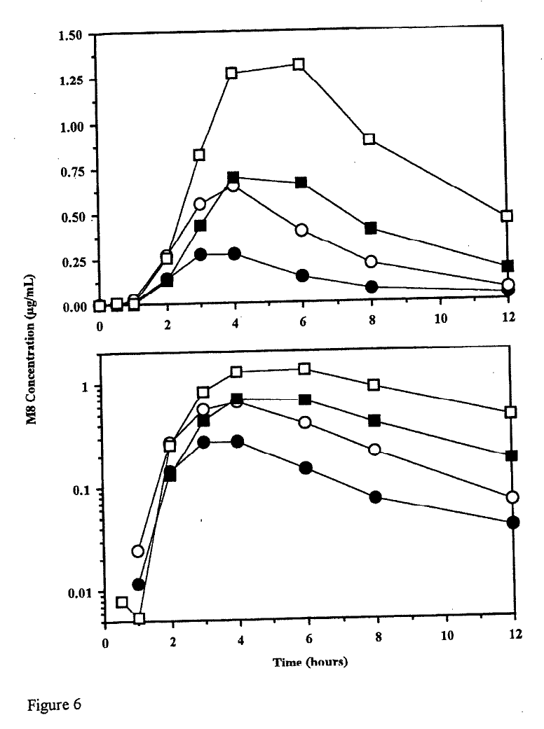

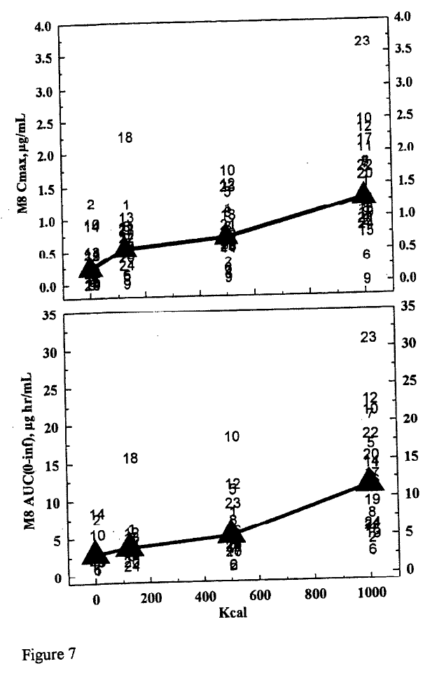

Mean plasma M8 concentration-time profiles for each treatment are depicted in FIG. 6. Mean M8 pharmacokinetic parameter values are presented in Table 6 along with ratios and confidence intervals. Individual Cmax and AUC(0-∞) values are illustrated in FIG. 7.

Effect of Caloric Content

This example shows that food intake has a marked effect on nelfinavir pharmacokinetics with the highest levels achieved after the greatest food intake. AUC values increased 3-5-fold over those in the fasting state by administering nelfinavir with meals containing 500-1000 kcal and 20-50% fat.

The metabolite, M8, plasma concentrations generally tracked those of nelfinavir. Based on the area under the plasma concentration-time profile from time zero extrapolated to infinite time (AUC (0-∞)) values, the bioavailability of M8 was 1.3-, 1.8 and 4.1-fold higher with increasing caloric intake relative to fasting. In the fed state the M8 AUC/nelfinavir AUC ranged from 15-21%.

The percentage of M8 relative to nelfinavir remained the same between the fed and fasted administration methods.

TABLE 6
Summary of M8 Pharmacokinetic Parameter Values Following
Administration of 1250-mg Nelfinavir Oral Doses to Fasting
Subjects (Reference), with a Low Calorie/Low Fat Meal,
with a Moderate Calorie/Low Fat Meal, and with a
High Calorie/High Fat Meal.

| | Least-Squares Mean Values | | | |
|---|---|---|---|---|
| | | with | | 90% |
| | Fasting | Meal | | Confidence |
| Parameter | (Reference) | (Test) | Ratio | Interval |

Low Calorie/Low Fat/Low Protein Meal

| | | | | |
|---|---|---|---|---|
| Cmax, μg/mL | 0.228^{a} | 0.533^{d} | 234 | 165 to 333 |
| tmax, hr | 3.45^{b} | 3.88^{e} | 113 | Not Applicable |
| AUC(0-tlqc), | 0.655^{a} | 2.01^{d} | 343 | 207 to 568 |
| μg · hr/mL | | | | |
| AUC(0-∞), | 2.81^{c} | 3.66^{f} | 130 | 88.3 to 192 |
| μg · hr/mL | | | | |
| t1/2, hr | 2.68^{c} | 2.40^{f} | 89.4 | 65.2 to 114 |

Moderate Calorie/Low Fat/Moderate Protein Meal

| | | | | |
|---|---|---|---|---|
| Cmax, μg/mL | 0.228^{a} | 0.693^{a} | 304 | 216 to 428 |
| tmax, hr | 3.45^{b} | 4.49^{a} | 130 | Not Applicable |
| AUC(0-tlqc), | 0.655^{a} | 3.46^{a} | 529 | 323 to 865 |
| μg · hr/mL | | | | |
| AUC(0-∞), | 2.81^{c} | 5.11^{e} | 182 | 126 to 263 |
| μg · hr/mL | | | | |
| t1/2, hr | 2.68^{c} | 2.96^{e} | 110 | 87.3 to 133 |

High Calorie/High Fat/High Protein Meal

| | | | | |
|---|---|---|---|---|
| Cmax, μg/mL | 0.228^{a} | 1.28^{g} | 562 | 401 to 787 |
| tmax, hr | 3.45^{b} | 5.01^{a} | 145 | Not Applicable |
| AUC(0-tlqc), | 0.655^{a} | 7.58^{g} | 1157 | 712 to 1878 |
| μg · hr/mL | | | | |
| AUC(0-∞), | 2.81^{c} | 11.5^{a} | 408 | 285 to 586 |
| μg · hr/mL | | | | |
| t1/2, hr | 2.68^{c} | 3.90^{a} | 145 | 123 to 168 |

^{a}N = 22;
^{b}N = 17;
^{c}N = 10;
^{d}N = 21;
^{e}N = 20;
^{f}N = 18;
^{g}N = 23
Ratio = Ratio of treatment mean values, expressed as a percentage (100% × test/reference).
90% Confidence Interval = 90% confidence interval estimate for the ratio (test/reference) of treatment mean values, expressed as a percentage of the reference mean.

### EXAMPLE 2

### Evaluation of Fat on Nelfinavir Bioavailability

A study was conducted as a phase 1, randomized, open-label, crossover 3×3 study, designed to evaluate the impact of a fixed kilocalorie meal at 20% and 50% fat content on single-dose pharmacokinetic parameters of the nelfinavir 250 mg tablet formulation in normal, healthy volunteers.

Methods

Subjects were dosed with 1250 mg of nelfinavir 3 times at one-week intervals and 24-hour PK profiles were collected following each of the doses. Each subject was assigned three meals with different fat contents prior to dosing (fasting, 500kcal with 20% fat, 500 kcal with 50% fat) using a Latin square design.

Twenty-four subjects entered the study and twenty-two subjects completed it. Each subject received the following treatment in random order on Days 1, 8 and 15: 5×250 mg nelfinavir tablets with fasting; 5×250 mg nelfinavir tablets, with a meal comprising 500Kcal/20% fat; and 5×250 mg nelfinavir tablets with a meal having 500 Kcal/50% fat.

The moderate calorie/low fat meal consisted of 500 Kcal with 20% fat (11.3 grams of fat). The moderate calorie/high fat meal consisted of 500 Kcal with 50% fat (27.8 grams of fat).

Subjects were administered nelfinavir 1250-mg (five 250-mg tablets) on the morning of the pharmacokinetic evaluations. The nelfinavir terminal half-life (t½) in plasma is typically 3.5 to 5 hours. To ensure clearance of nelfinavir between evaluations, PK evaluations were performed on Days 1, 8 and 15 such that there would be a 7-day washout between doses. Subjects participated on an outpatient basis; however, subjects were admitted to the in-patient facility the evening prior to each PK evaluation and remained in the in-patient facility for approximately 16 hours post-dosing. The subjects returned the next morning (8 hours later) for their last 24 hr. pharmacokinetic blood draw. All pharmacokinetic evaluations were performed in the in-patient facility. Blood samples were collected and analyzed for plasma concentrations of nelfinavir and M8.

For the fasting evaluation, subjects were required to complete an overnight fast of at least 10 hours prior to dosing in the morning. For the fed PK evaluations, subjects were required to complete an overnight fast of at least 10 hours, prior to receiving the protocol-specific standardized breakfast meal. Subjects were given 30 minutes to complete their standardized breakfast meal. Dosing was performed in the morning, immediately following the subject's completion of the standardized breakfast meal and after the subject's predose PK specimen had been collected. Subjects could not ingest water 1 hour prior to or 1 hour after dosing. A standardized lunch was given at least 4 hours after the morning dose and a standardized dinner was given at least 10 hours after the morning dose.

Actual sampling times were used for all data evaluation. Mean Cmax and AUC values were calculated as the antilogs of least-squares mean log-transformed values (analogous to geometric means). Ratios and confidence intervals for Cmax and AUC values are also based on log-transformed values. Mean values for all other pharmacokinetic parameters are least-squares means. Ratios and confidence intervals for these parameters are based on untransformed values.

As in Example 1, healthy volunteers of any race and either gender, 18 to 60 years of age (inclusive), were used in the study. Volunteers were chosen who had a body mass index (BMI) between 18 to 31 kg/m² (inclusive), and who were seronegative for human immunodeficiency virus (HIV) −1/HIV −2. Females were required to be not pregnant and be using a reliable barrier method of birth control, have been surgically sterilized, or be postmenopausal.

Pharmacokinetics

Blood samples, 5 mL each, were collected as in Example 1. The timing of each sample collection was as follows: Predose and at 0.5, 1, 2, 3, 4, 6, 8, 12, 16 and 24 hours postdose on Days 1, 8, and 15. All blood samples were kept at 40 C (using either wet ice or cryoblock) until centrifugation. Blood samples were centrifuged within 1 hour of collection, at 3000 rpm (approximately 2619×g) for 15 minutes, to separate the plasma. The plasma samples were split evenly into 2 aliquots and stored in appropriately labeled polypropylene transport tubes. Plasma was stored frozen at 20° C. or lower until analysis.

Sample analysis for nelfinavir and M8 in plasma is summarized in Table 7.

TABLE 7
Summary of Sample Analysis for Nelfinavir and M8 in Human Plasma
(Example 2)

| | |
|---|---|
| Method Description | Plasma (Sodium Heparin) |
| Matrix | HPLC |
| Type of Method | None |
| Deviations From Validated Method | 250 μL |
| Sample Volume | ALD-126462 |
| Internal Standard | |

Study Assay Performance

| | Analytical Range | | Quality Control Samples | |
|---|---|---|---|---|
| | Lower Limit | Upper Limit | Precision | Accuracy |
| Analyte | (LLOQ) | (ULOQ) | (% CV) | (% RE) |
| Nelfinavir | 0.0500 μg/mL | 10.0 μg/mL | ≦9.59% | 3.76 to |
| | | | | 6.58% |
| MB (AG-1402) | 0.0500 μg/mL | 10.0 μg/mL | ≦2.83% | 4.29 to |
| | | | | 6.64% |

Sample Handling

| | |
|---|---|
| Storage Conditions | −20° C. |
| Stability Under Storage Conditions | 656 days |
| Stability ≧ Longest Time From Collection to | Yes |
| Analysis | |

Pharmacokinetic parameter values were calculated from plasma nelfinavir and M8 concentration-time data using standard noncompartmental pharmacokinetic methods as in Example 1.

Log-transformed nelfinavir AUC was the primary parameter used in the evaluation of the potential effect of fat content of the meals on nelfinavir pharmacokinetics. Secondary parameters included in this analysis were nelfinavir terminal half-life (t2), time to maximum plasma concentration (tmax), and log-transformed Cmax, as well as M8 pharmacokinetic parameters. Parameter values were evaluated by analysis of variance (ANOVA) using a model incorporating sequence, subject within sequence, period, and treatment effects. Statistical tests were performed using the Type III sum of squares derived using WinNonlin Pro Version 2.1. Least-squares treatment mean values were determined for each parameter.

Results from ANOVA were used to calculate 90% confidence intervals for the ratio (test/reference) least-squares treatment mean values, where administration of single nelfinavir doses fasting was the reference treatment. Confidence intervals were calculated using WinNonlin Pro Version 2.1. Confidence intervals were used as an aid in data interpretation. Descriptive statistics of nelfinavir Cmax and AUC were examined to determine the effect of meals of various fat content on the variability of these parameter values.

Plasma concentrations of nelfinavir and M8 were measured by validated high-performance liquid chromatography (HPLC) methods. Pharmacokinetic parameters were determined from plasma concentration-time data using standard noncompartmental methods.

Statistical Methods

Log-transformed nelfinavir area under the plasma concentration-time profile (AUC) values was the primary parameter analyzed to determine the effect of fat content of meals on nelfinavir pharmacokinetics. The 90% confidence intervals for the ratios of test (with test meal) to reference (fasting) least-squares mean AUC as well as maximum observed plasma concentration (Cmax) values were calculated using log-transformed data and expressed as a percentage of the reference mean. The relationship between nelfinavir exposure and fat content of the meals was examined.

Pharmacokinetic Results

Nelfinavir pharmacokinetic parameter values following administration of 5×250-mg nelfinavir tablets to fasting subjects (reference), during a moderate calorie/low fat meal, and during a moderate calorie/high fat meal are summarized in the following Table 8. 0

TABLE 8
Nelfinavir Pharmacokinetic Parameter Values, Example 2

| | Least-squares Mean Values | | | |
|---|---|---|---|---|
| | Fasting | With Meal | | 90% Confidence |
| Parameter | (Reference) | (Test) | Ratio | Interval |

Test = Moderate Calorie/Low Fat

| | | | | |
|---|---|---|---|---|
| N | 22 | 22 | | |
| Cmax, μg/mL | 1.63 | 4.04 | 248 | 199 to 308 |
| tmax, hr | 2.42 | 4.19 | 173 | Not Applicable |
| AUC(0-tlqc), | 10.0 | 32.6 | 325 | 252 to 419 |
| μg hr/mL | | | | |
| AUC(0-∞), | 10.7 | 32.7 | 305 | 239 to 389 |
| μg hr/mL | | | | |
| t1/2, hr | 4.10 | 3.08 | 75.0 | 46.5 to 104 |

Test = Moderate Calorie/High Fat

| | | | | |
|---|---|---|---|---|
| N | 22 | 22 | | |
| Cmax, μg/mL | 1.63 | 6.16 | 378 | 304 to 370 |
| tmax, hr | 2.42 | 4.48 | 186 | Not Applicable |
| AUC(0-tlqc), | 10.0 | 52.6 | 524 | 407 to 676 |
| μg hr/mL | | | | |
| AUC(0-∞), | 10.7 | 54.6 | 508 | 398 to 649 |
| μg hr/mL | | | | |
| t1/2, hr | 4.10 | 3.43 | 83.7 | 55.2 to 112 |

Definitions of terms used in the table include: “Ratio” is the ratio of treatment mean values, expressed as a percentage (100% x test/reference). “90% Confidence Interval” is the 90% confidence interval estimate for the ratio (test/reference) of treatment mean values, expressed as a percentage of the reference mean.

Administration of nelfinavir with meals of similar caloric content and 20% and 50% fat content resulted in longer time to maximum plasma concentration (tmax) values and higher Cmax values. Mean tmax values were approximately 2 hours longer when administered with meals of 20% and 50% fat content, relative to that in fasting subjects. Mean Cmax values were approximately 2.5- and 3.8-fold higher in meals of 20% and 50% fat content, respectively. Based on area under the plasma concentration-time profile from time zero extrapolated to infinite time (AUC(0-∞)) values, the bioavailability of nelfinavir was approximately 3- and 5-fold higher following administration of meals containing 20% and 50% fat content, respectively, relative to that in fasting subjects. Fat content did not have a profound effect on nelfinavir terminal half-life (t2) values. Nelfinavir elimination t½ values following administration to fasting subjects and with test meals were similar, averaging approximately 4 hours.

Administration of nelfinavir with meals of 20% and 50% fat content resulted in lower variability in plasma concentrations, relative to that in fasting subjects. Values for % coefficient of variation (CV) for AUC(0-∞) were 75% in fasting subjects and 48% and 43% in subjects receiving the test meals containing 20% and 50% fat, respectively.

M8 plasma concentrations generally tracked those of nelfinavir. Mean M8 AUC(0-∞) were 3- and 6.8-fold higher following administration of test meals containing 20% and 50% fat, respectively, relative to that in fasting subjects.

Results

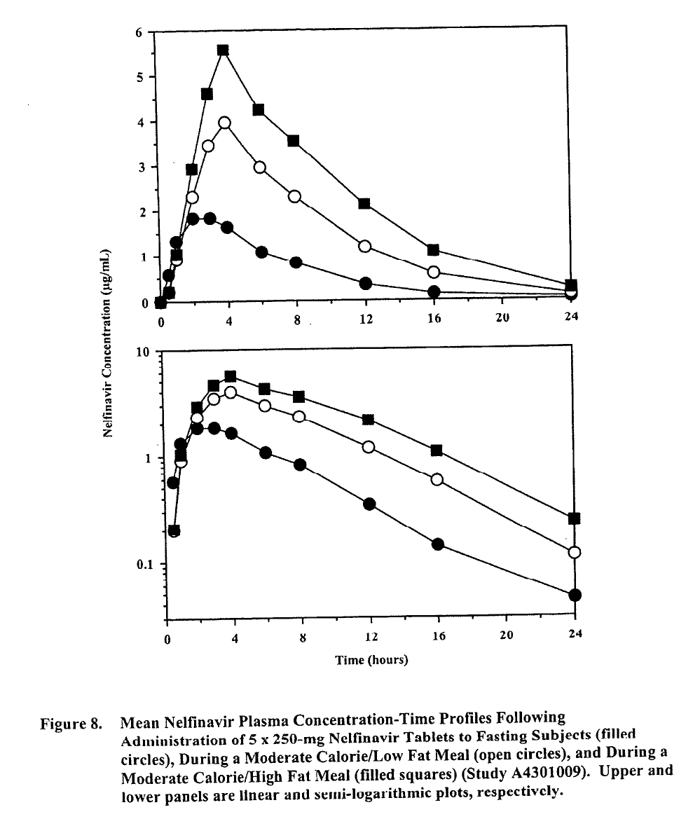

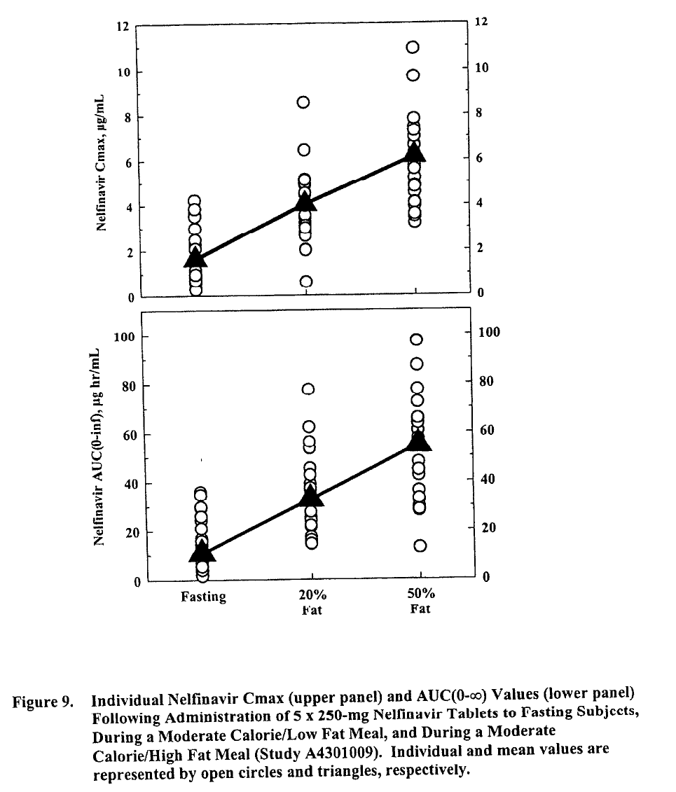

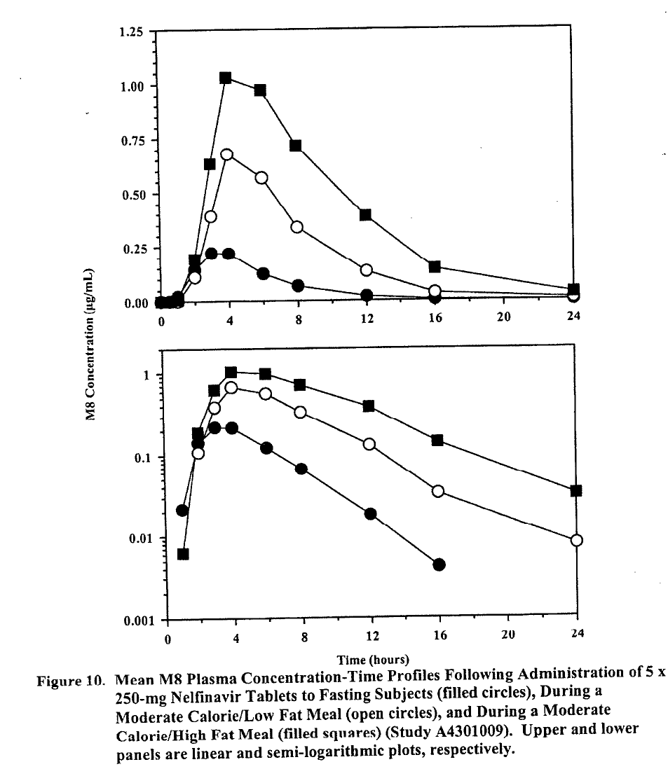

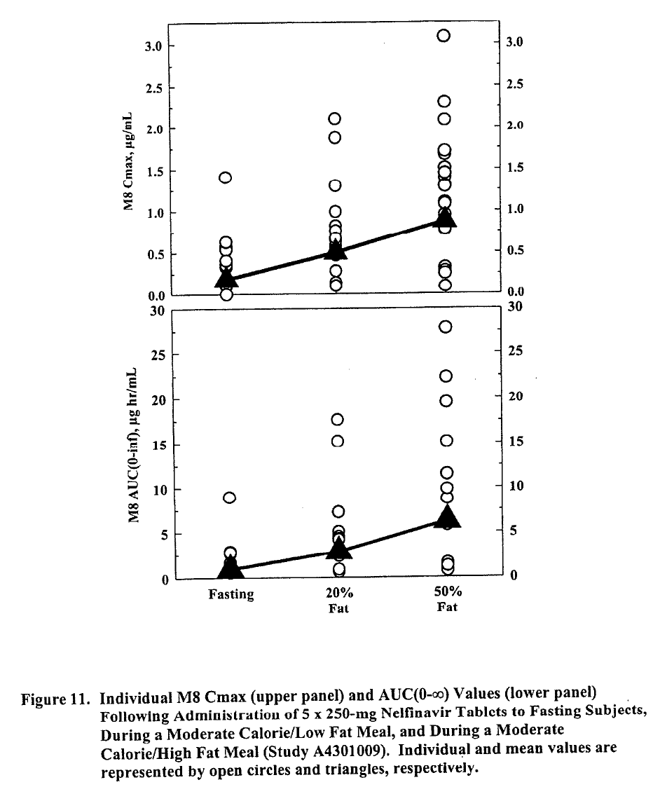

Mean plasma nelfinavir concentration-time profiles for each treatment are depicted in FIG. 8. Mean nelfinavir pharmacokinetic parameter values in the comparison of nelfinavir administration with test meals relative to those to fasting subjects are presented in Table 9, along with ratios and confidence intervals. Individual Cmax and AUC values are illustrated in FIG. 9. Corresponding data for M8 concentration—time profiles and also M8 Max and AUC are presented in FIGS. 10 and 11, respectively.

TABLE 9
Summary of Nelfinavir Pharmacokinetic Parameter Values Following
Administration of 5 × 250-mg Nelfinavir Tablets to Fasting Subjects
(Reference), During a Moderate Calorie/Low Fat Meal, and During a
Moderate Calorie/High Fat Meal (Study 2)

| | Least-squares Mean Values | | | |
|---|---|---|---|---|
| | Fasting | With Meal | | 90% Confidence |
| Parameter | (Reference) | (Test) | Ratio | Interval |

Test = Moderate Calorie/Low Fat

| | | | | |
|---|---|---|---|---|
| N | 22 | 22 | | |
| Cmax, μg/mL | 1.63 | 4.04 | 248 | 199 to 308 |
| tmax, hr | 2.42 | 4.19 | 173 | Not Applicable |
| AUC(0-tlqc), | 10.0 | 32.6 | 325 | 252 to 419 |
| μg hr/mL | | | | |
| AUC(0-∞), | 10.7 | 32.7 | 305 | 239 to 389 |
| μg hr/mL | | | | |
| t1/2, hr | 4.10 | 3.08 | 75.0 | 46.5 to 104 |

Test = Moderate Calorie/High Fat

| | | | | |
|---|---|---|---|---|
| N | 22 | 22 | | |
| Cmax, μg/mL | 1.63 | 6.16 | 378 | 304 to 370 |
| tmax, hr | 2.42 | 4.48 | 186 | Not Applicable |
| AUC(0-tlqc), | 10.0 | 52.6 | 524 | 407 to 676 |
| μg hr/mL | | | | |
| AUC(0-∞), | 10.7 | 54.6 | 508 | 398 to 649 |
| μg hr/mL | | | | |
| t1/2, hr | 4.10 | 3.43 | 83.7 | 55.2 to 112 |

Parameters are described in Table 16
Ratio = Ratio of treatment mean values, expressed as a percentage (100% × test/reference)
90% Confidence = 90% confidence interval estimate for the ratio (test/reference) of treatment mean values, expressed as a percentage of the reference mean

This example indicates that fat intake has a marked effect on nelfinavir pharmacokinetic parameters after a single dose exposure. AUC values increased 3.2 fold with a 500 kcal, 20% fat breakfast and 5.2 fold with the same Kcal but 50% fat when compared to the fasting AUC. These values were similar to the values previously determined for a 500 Kcal, 20% fat breakfast and a 1000 Kcal, 50% fat breakfast. Thus fat content in meals affects nelfinavir PK in addition to its Kcal content suggesting a plateau effect for Kcal content.

The discovery that 500 kcal and 1000 kcal yield the same fold increase in plasma exposure if they are administered as 50% fat has important implications for optimal use of nelfinavir. A 500kcal/50% fat meal can be delivered as 3.5-4 ounces of roasted peanuts, less than one cup of canned coconut cream, or a variety of American breakfast fast foods. Also, the fat dependence allows the development of a formulation of nelfinavir with fat that would enhance compliance with optimal administration of the medication. This example also shows that M8 concentrations rose with increasing fat intake but that the percentage of M8 relative to nelfinavir remained the same, around 10%.

TABLE 10
Summary of Example 2: Pharmacokinetic Parameter Values

| | | Kcal/Fat % | Kcal/Fat % |
|---|---|---|---|
| PK Parameter | Fasting | 500/20% | 500/50% |
| Nelfinavir AUC24, mg · hr/mL | 10.0 | 32.6 (3.2X) | 52.6 (5.2X) |
| (×fasting) | | | |
| 90% Cl, ×fasting¹ | | 2.5-4.1X | 4.1-6.8X |
| Nelfinavir AUC∞, mg · hr/mL | 10.7 | 32.7 (3.0X) | 54.6 (5.1X) |
| (×fasting) | | | |
| 90% Cl, ×fasting | | 2.4-3.9X | 4.0-6.5X |
| Nelfinavir Cmax, mg/mL, | 1.63 | 4.0 (2.5X) | 6.2 (3.8X) |
| (×fasting) | | | |
| 90% Cl, ×fasting | | 2.0-3.1X | 3.0-4.7X |
| M8 AUC∞/ | 8.6 | 8.7 | 11.4 |
| Nelfinavir AUC∞(%) | | | |

The 90% confidence interval (90% CI) is calculated for the ratio above the CI. The ratio of plasma levels of nelfinavir metabolite to nelfinavir is expressed as “M8 AUC∞/nelfinavir AUC∞ (×100).

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

We claim:

1. A method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily the nelfinavir is administered with food and the food comprises more than 800 kcal.

2. The method of claim 1, wherein the food comprises more than about 900 kcal.

3. The method of claim 1, wherein the food comprises more than about 1000 kcal.

4. The method of claim 1, wherein the administration of nelfinavir occurs between 30 minutes prior to and two hours after consumption of food.

5. The method of claim 1, wherein the administration of nelfinavir occurs between 30 minutes prior to and one hour after consumption of food.

6. The method of claim 1, wherein the administration of nelfinavir occurs at about the same time as the consumption of food.

7. The method of claim 1, wherein nelfinavir is administered at least twice daily for at least two weeks and at least twice daily nelfinavir is administered with food and the food comprises more than 800 kcal at each administration.

8. The method of claim 1, wherein the food comprises between about 40% fat and about 50% fat by energy content.

9. The method of claim 1, wherein the food comprises between about 50% fat and about 60% fat by energy content.

10. The method of claim 1, wherein the food comprises between about 60% fat and about 70% fat by energy content.

11. The method of claim 1, wherein the food comprises between about 70% fat and about 80% fat by energy content.

12. The method of claim 1, wherein the food comprises between about 80% fat and about 90% fat by energy content.

13. The method of claim 1, wherein the food comprises between about 90% fat and about 100% fat by energy content.

14. The method of claim 1, wherein the food comprises more than 40% fat by energy content.

15. The method of claim 1, wherein the food comprises more than about 50% fat by energy content.

16. The method of claim 1, wherein the food comprises more than about 60% fat by energy content.

17. The method of claim 1, wherein the food comprises more than about 70% fat by energy content.

18. The method of claim 1, wherein the food comprises more than about 80% fat by energy content.

19. The method of claim 1, wherein the food comprises more than about 90% fat by energy content.

20. The method of claim 1, wherein the food comprises from 36 9 to 55 g fat.

21. The method of claim 1, wherein the food comprises from 40 9 to 55 g fat.

22. The method of claim 1, wherein the food comprises at least about 55 g fat.

23. The method of claim 1, wherein the area under the curve from time zero extrapolated to infinite time (AUC(0-∞)) after nelfinavir administration with food is at least about 3-fold greater than the AUC(0-∞) after administration in the fasted state.

24. The method of claim 23, wherein the AUC(0-∞) after nelfinavir administration with food is at least about 5-fold greater than the AUC(0-∞) after administration in the fasted state.

25. The method of claim 1, wherein the mammal is not receiving ritonavir, saquinavir or lopinavir or a stereoisomer, solvate, salt, or prod rug thereof.

26. A method of treating human immunodeficiency virus (HIV) in a mammal comprising administering orally to a mammal in need thereof a therapeutically effective amount of nelfinavir or pharmaceutically acceptable salt or solvate thereof in a pharmaceutical composition taken with food, wherein the food comprises at least about 500 kcal and at least about 50% fat by energy content.

27. The method of claim 26 wherein the administration of nelfinavir occurs between 30 minutes prior to and two hours after consumption of food.

28. The method of claim 26, wherein the administration of nelfinavir occurs between 30 minutes prior to and one hour after consumption of food.

29. The method of claim 26, wherein the administration of nelfinavir occurs at about the same time as the consumption of food.

30. The method of claim 26, wherein the food comprises between about 50% fat and about 60% fat by energy content.

31. The method of claim 26, wherein the food comprises between about 60% fat and about 70% fat by energy content.

32. The method of claim 26, wherein the food comprises between about 70% fat and about 80% fat by energy content.

33. The method of claim 26, wherein the food comprises between about 80% fat and about 90% fat by energy content.

34. The method of claim 26, wherein the food comprises between about 90% fat and about 100% fat by energy content.

35. The method of claim 26, wherein the food comprises more than about 60% fat by energy content.

36. The method of claim 26, wherein the food comprises more than about 70% fat by energy content.

37. The method of claim 26, wherein the food comprises more than about 80% fat by energy content.

38. The method of claim 26, wherein the food comprises more than about 90% fat by energy content.

39. The method of claim 26, wherein the food comprises from 36 g to 55 g fat.

40. The method of claim 26, wherein the food comprises from 40 g to 55 g fat.

41. The method of claim 26, wherein the food comprises at least about 55 g fat.

42. The method of claim 26, wherein the food comprises at least about 600 kcal.

43. The method of claim 26, wherein the food comprises at least about 700 kcal.

44. The method of claim 26, wherein the food comprises at least about 800 kcal.

45. The method of claim 26, wherein the food comprises at least about 900 kcal.

46. The method of claim 26, wherein the food comprises at least about 1000 kcal.

47. The method of claim 26, wherein the area under the curve from time zero extrapolated to infinite time (AUC(0-∞)) after nelfinavir administration with food is at least about 3-fold greater than the AUC(0-∞) after administration in the fasted state.

48. The method of claim 47, wherein the AUC (0-∞) after nelfinavir administration with food is at least about 5-fold greater than the AUC (0-∞) after administration in the fasted state.

49. The method of claim 26, wherein the mammal is not receiving ritonavir, saquinavir or lopinavir or a stereoisomer, solvate, salt, or prod rug thereof.

50. A method of treating human immunodeficiency virus (HIV) in a mammal comprising administering to a mammal in need thereof a therapeutically effective amount of nelfinavir or a pharmaceutically acceptable salt or solvate thereof in a pharmaceutical composition at least once daily for at least two weeks, wherein at least once daily nelfinavir is taken with food and the food comprises more than about 500 kcal and more than about 50% fat by energy content.

51. The method of claim 50, wherein the administration of nelfinavir occurs between 30 minutes prior to and two hours after consumption of food.

52. The method of claim 50, wherein the administration of nelfinavir occurs between 30 minutes prior to and one hour after consumption of food.

53. The method of claim 50, wherein the administration of nelfinavir occurs at about the same time as the consumption of food.

54. The method of claim 50, wherein nelfinavir is administered at least twice daily for at least two weeks and at least twice daily nelfinavir is administered with food and the food comprises more than 500 kcal and more than about 50% fat by energy content at each administration.

55. The method of claim 50, wherein the food comprises more than about 600 kcal.

56. The method of claim 50, wherein the food comprises more than about 700 kcal.

57. The method of claim 50, wherein the food comprises more than about 900 kcal.

58. The method of claim 50, wherein the food comprises more than about 1000 kcal.

59. The method of claim 50, wherein the food comprises between about 50% fat and about 60% fat by energy content.

60. The method of claim 50, wherein the food comprises between about 60% fat and about 70% fat by energy content.

61. The method of claim 50, wherein the food comprises between about 70% fat and about 80% fat by energy content.

62. The method of claim 50, wherein the food comprises between about 80% fat and about 90% fat by energy content.

63. The method of claim 50, wherein the food comprises between about 90% fat and about 100% fat by energy content.

64. The method of claim 50, wherein the food comprises more than about 60% fat by energy content.

65. The method of claim 50, wherein the food comprises more than about 70% fat by energy content.

66. The method of claim 50, wherein the food comprises more than about 80% fat by energy content.

67. The method of claim 50, wherein the food comprises more than about 90% fat by energy content.

68. The method of claim 50, wherein the food comprises from 36 g to 55 g fat.

69. The method of claim 50, wherein the food comprises from 40 g to 55 g fat.

70. The method of claim 50, wherein the food comprises at least about 55 g fat.

71. The method of claim 50, wherein the mammal is not receiving ritonavir, saquinavir or lopinavir or a stereoisomer, solvate, salt or prodrug thereof.

72. The method of claim 50, wherein the area under the curve from time zero extrapolated to infinite time (AUC(0-∞)) after nelfinavir administration with food is at least about 3-fold greater than the AUC(0-∞) after administration in the fasted state.

73. The method of claim 72, wherein the AUC (0-∞) after nelfinavir administration with food is at least about 5-fold greater than the AUC (0-∞) after administration in the fasted state.

74. The method of claim 50, wherein the mammal is not receiving ritonavir, saquinavir or lopinavir or a stereoisomer, solvate, salt, or prod rug thereof.

75. A kit comprising a therapeutically effective oral dose of nelfinavir and a printed material comprising instructions for administering the dose with food comprising at least 800 kcal in a high-fat meal.

76. The kit of claim 75, wherein the label further comprises instructions for administering the dose with food comprising at least 50% fat by energy content.

77. The kit of claim 75, wherein the high-fat meal is recited to comprise more than about 36 g of fat.

78. A therapeutic composition for the treatment of human immunodeficiency virus (HIV) in a mammal comprising fat and a therapeutically effective amount of nelfinavir in a weight ratio of at least about 25 fat: 1 nelfinavir.

79. The composition of claim 78, wherein the weight ratio is greater than about 30 fat:1 nelfinavir.

80. The composition of claim 78, wherein the amount of nelfinavir is between about 100 mg and about 1500 mg.
